(19) United States
(12) Patent Application Publication
(10) Pub. No.: US 2024/0215995 A1
(43) Pub. Date: Jul. 4, 2024

(54) APPARATUSES, SYSTEMS AND METHODS FOR BLOOD CLOT AND TUMOR REMOVAL

(71) Applicant: Neuroventis, La Jolla, CA (US)

(72) Inventors: Thomas Afzal, Coronado, CA (US); Carl Lauryssen, Austin, TX (US)

(21) Appl. No.: 17/803,181

(22) Filed: Dec. 2, 2021

Related U.S. Application Data
(60) Provisional application No. 63/205,330, filed on Dec. 2, 2020.

(51) Int. Cl. A61B 17/22; A61B 17/00; A61B 17/34; A61B 90/00

(52) U.S. Cl. CPC A61B 17/22; A61B 17/3423; A61B 90/361; A61B 2017/00017; A61B 2017/00084; A61B 2017/22079; A61B 2017/3425; A61B 2017/3445; A61B 2090/3612; A61B 2217/005; A61B 2217/007

(57) ABSTRACT

Apparatuses, systems and methods that provide to the neurosurgeon all of the features and functions needed for the entire surgical procedure in a fully integrated package that will allow for efficient and effective remove of the clot or tumor without the need for a craniotomy—from initial assessment of the clot or tumor, its size, location and status, to planning of the surgical procedure (from scalp incision to trajectory and depth of insertion of the cannula,) to controlled addition and removal of temperature-controlled fluid to the clot or tumor site, to precise and controlled removal of the clot or tumor, to confirmation of desired removal, to cauterization—and all done with real time visualization on adjacent video monitors or augmented reality displays that will allow the surgeon to “see” inside the brain.

### RELATED PROVISIONAL APPLICATIONS

This utility application relates to, updates, and incorporates by reference provisional patent application No. 63/205,330, filed Dec. 2, 2020, and claims the benefit of the filing date of that provisional application.

### FIELD OF THE INVENTION

The inventive concepts herein described, depicted and claimed relate generally to apparatuses, systems and methods for the evacuation of blood clots, particularly for neurosurgical evacuation and aspiration procedures such as the treatment of intracranial hemorrhage, and for the removal of tumors from the brain.

### BACKGROUND AND RELATED ART

Intracranial hemorrhage (ICH) is bleeding that occurs inside the skull. This can be the result of head injury, or more often, hemorrhagic stroke. There are two types of such strokes: intracerebral hemorrhage, in which a blood vessel inside the brain bursts and leaks blood into and around surrounding brain tissue; and subarachnoid hemorrhage, in which there is bleeding in the area between the brain and the tissue covering the brain, known as the subarachnoid space. Subarachnoid hemorrhage is most often caused by a burst aneurysm.

An aneurysm is an abnormal bulge or ballooning in the wall of a blood vessel, which can occur in any part of the body, but is potentially most serious, harmful and deadly when it occurs in the brain. Aneurysms have been called a silent killer, because they usually do not cause any symptoms before the rupture, even if the pre-ruptured aneurysm is quite large.

An ICH is serious as it may crush adjacent delicate brain tissue, limit the brain's blood supply and cause potentially deadly brain herniation where parts of the brain are squeezed past structures in the skull. Hemorrhaged blood may collect and congeal into a blood clot, potentially impacting circulation in the brain, resulting in cell death. In many cases, unless treated promptly and successfully, a ruptured aneurysm in the brain is deadly. Therefore, removing or reducing hemorrhaged blood and/or blood clots in the brain as quickly and precisely as possible (so as not to damage adjacent healthy brain cells) is crucial to increasing the likelihood of patient recovery.

Intracranial hemorrhage is deadly. The estimated survival rate for hemorrhagic stroke is less than 30%, and most who die, die quickly, as the 30-day survival rate is less than 50%. Approximately one-half of these patients who die within the first month die within 24 hours after onset. Even if the stroke does not cause death quickly, it remains deadly. Reported data indicates that over half of those patients who initially survive died from complications of the stroke within a year after onset. ICH is typically listed as one of the top five causes of death in the United States, year in and year out.

Of those patients who survive for months or years after the stroke, over two-thirds suffer long-term neurological problems and require dependent care. The potentially long-lasting deleterious effects of an ICH include some or all of the following: inability to move part(s) of the patient's body (paralysis); numbness or weakness in part(s) of the body; difficulty swallowing; vision impairment or total loss; hearing impairment or total loss; difficulty in speaking, communicating and comprehension; personality change and/or emotional problems; and/or confusion, memory loss, or impaired judgment. Any and all of these can also present safety risks to the afflicted person and his or her surroundings. Many who have survived the stroke are no longer able to care for themselves, with over 60% requiring full-time dependent care.

The impact of an ICH on the patient can be and often is devastating, and the resultant strain on family members, financially and otherwise, who must cope with seeing their loved one die or suffer long term physical and mental impairment, and other health issues, and who also must provide or provide for dependent care, can also cause significant collateral damage within the family.

Sadly, the incidence of ICH is large, and is growing, as the so-called baby boomer generation is aging. According to recently released U.S. Census data, the 65-and-older population in the United States grew by over a third (34.2% or 13,787,044) during the past decade, and by 3.2% (1,688,924) from 2018 to 2019 alone. By the year 2030, the entire still-then-living baby boomers will be over age 65. Even today, there are an estimated 170,000 to 200,000 incidents of hemorrhagic stroke each year in the United States.

As this narrative demonstrates, ICH is a serious and growing risk to human health. It is also a condition that demands immediate medical treatment, which can greatly increase the survival rate, and also limit the lasting deleterious effects of the stroke.

Depending on the size and location of the ICH or blood clot, its removal may be performed via aspiration of the clot, such as with a catheter, but most often by means of a craniotomy. Surgery is generally required if the ICH is greater than 3 cm in diameter. Current “standard of care” treatment for such conditions requires a craniotomy. This literally provides a “window” to the brain, allowing visual and physical access by the neurosurgeon to the brain, and allowing for continued direct visualization and access during the critical period of treatment when such continued access may be desirable or necessary.

During a craniotomy, the neurosurgeon cuts and entirely removes a significant portion of the skull (up to one half of the skull) to provide physical and visual access for directly visualizing blood clots to distinguish the clot from brain tissue and to be able to safely remove the blood clot without damaging surrounding brain tissue, which is a significant risk when performing such procedures designed to remove the clot, as the same techniques and tools used to remove the clot can also damage surrounding brain tissue.

It is also important in some cases not to remove too much of the blood clot. The clotting blood is the body's way of stopping the continued flow of blood from the ruptured vessel. The clot is in a way the body's way of plugging the hole, so to speak. Remove too little of the clot, and the pressure on surrounding tissue is not sufficiently alleviated. Remove too much of the clot, and the bleeding resumes which could make the patient's condition worse than before. If removal of the entire clot is required, then cauterization of the vessel may be required. This too presents a significant risk to surrounding healthy brain tissue if not precisely controlled. Thus, such surgeries are high risk, and are typically accompanied by a long period of recovery.

The ability of the neurosurgeon to be able to “see” precisely what is going on in the brain during the neurosurgery is therefore of critical importance. Such visualization is possible with a craniotomy, but unless the large portion of the skull is removed, the surgeon can be essentially “flying blind,” or nearly so. In this regard, neuronavigation is a tool that is often used by neurosurgeons. In general, neuronavigation is a set of computer-assisted technologies used to guide or “navigate” within the confines of the skull. The ability to relate the position of a real surgical instrument in the surgeon's hand or the microscope's focal point to the location of the imaged pathology, updated in “real time” in an “integrated operating room,” allows the surgeon attempting to remove a blot clot to “see” the precise location of the removal tool within the brain. This same situation also exists with respect to the remove of brain tumors found in the brain.

Current technology and clinical solutions need improvement to provide desired patient outcomes. Limitations for treatment available for patients affect mortality rate and quality of life for survivors (also for the quality of life for those who must provide dependent care for the survivors). Therefore, there is a need for improved methods and tools to provide ease of navigation, visualization, and removal of blood clots and tumors from the brain.

One such significant improvement would be a system in which the neurosurgeon can have real-time, accurate virtual visualization of the clot or tumor and the surrounding brain tissue (using MRI and CT scans) on video monitors in the operating room of the clot, the surrounding tissue, and using integrated neuronavigation, also the precise position of the surgical device that has been inserted into the brain, without having to first perform a craniotomy. This will be a very significant benefit to those hospitals and treatment facilities that do not have the requisite equipment on-hand to be able to determine the size and location of the clot such that the surgeons have no choice other than to perform a craniotomy, or to transport the patient to a facility that has such requisite equipment, which would involve a delay that could be very detrimental to the patient.

It would also be beneficial if a single insertion tool could be used for intracranial visualization of the clot or tumor, for providing for removal of the clot and for cauterization where that is desired, or for the insertion of other tools simultaneously while the clot removal device remains in-situ.

### SUMMARY OF INVENTIVE CONCEPTS

Some of the inventive concepts and advantages achieved thereby include a fully integrated system for assessment of and surgery to remove the ICH (or tumor) in a precise, controlled and minimally invasive manner and procedure. This will include using the patient's CT or MRI and standard neuronavigation system for planning the surgical approach; a software system that integrates with neuronavigation for planning and providing continued assessment of the ICH to be removed. In-situ ultrasound visualization technology is included to determine size, geometry, volume and density of ICH and correlate and communicate that for visualization on an integrated video monitor for real-time assessment by the neurosurgeon. The integrated software assesses volume of clot removed and remaining clot based on baseline of patients CT or MRI scan. The system also provides for secondary visualization by means of an inserted neuroendoscope for direct visualization. The system can break-up acute and subacute blood clot and provide controlled aspiration. The system integrates SMART sensor technology for pressure, temperature, and oxygenation feedback.

The apparatuses disclosed will employ an elongated cannula, which will be inserted through a small hole in the skull into the brain and into the clot. Embodiments of the cannula include one that is approximately 12 cm long with diameters ranging from 4 mm, 5 mm, 6 mm, 7 mm, 8 mm, 9 mm, 10 mm, 11 mm, and 12 mm and is capable of accommodating multiple lumen, for example, for the controlled delivery of fluid delivery and aspiration; for appropriate safety controls in which fluid delivery to the clot location in the brain cannot exceed the rate of fluid aspiration so as to avoid introducing increased fluid pressure in situ; it can provide cautery to seal bleeding vessels; and it can include a steerable/deflectable tip to navigate within the clot. The cannula can include a fixed scope for visualization or a scope element that can be retracted in-situ so that the surgeon can have a wider field of visualization of the clot (or tumor) without having the withdraw the cannula itself.

One embodiment of the system and method provides localized cooling and delivery of oxygenated fluid, which in turn provides advanced neuroprotection capability during treatment. The system is able to circulate continuous hyperosmotic hypothermic solution. This reduces cellular swelling and brain edema by increasing blood supply and stabilization of cellular membranes.

In another embodiment of the apparatuses, systems and methods disclosed, the integrated system will implement augmented reality. Augmented reality, in general, is an interactive experience of a real-world environment where the objects that reside in the real world are enhanced by computer-generated perceptual information, including visual. This generally involves three basic features: a combination of real and virtual worlds, real-time interaction, and accurate 3D registration of virtual and real objects.

Here, the integrated system would provide an augmented-reality view to the neurosurgeon of the patient's brain, and the location, shape and contours (among other things) on a real-time and instantaneous basis. The hardware components generally needed to implement augmented reality are: a processor, display, sensors and input devices. Various technologies are used in augmented reality rendering, including optical projection systems, monitors, handheld devices, and display systems, which are worn on the human body.

One such device, called a head-mounted display, is a display device worn on the forehead, such as a harness or helmet-mounted. These devices place images of both the physical world and virtual objects over the user's field of view. Modern devices of this type often employ sensors for six degrees of freedom monitoring that allow the system to align virtual information to the physical world and adjust accordingly with the user's head movements. Augmented reality displays included in the integrated systems disclosed can include devices resembling eyeglasses and goggles.

As mentioned above, the need for precision during the surgical procedure is of utmost importance. The neurosurgeon needs not only to be able to precisely “see” inside the brain to visualize the clots location and boundaries, but also to precisely guide the tools being inserted into the brain and into the clot so as to remove only that which is desired to be removed, without damage to surrounding material. This of course requires a very “steady hand” by the neurosurgeon. Even very slight unwanted movement of the inserted surgical tools can cause a less-than-desired result. Such unwanted, inadvertent movement of the surgical tool could be caused when the neurosurgeon turns his or her head to look at a video monitor or other image. To avoid the necessity of such movement, one embodiment of the apparatuses, systems and methods disclosed will include video goggles and/or smart glasses, in which the images (real and/or augmented) that the neurosurgeon needs or wants to view during the procedure can be displayed on the goggles or glasses such there is no need for the neurosurgeon to move his or her head at all during the critical portions of the procedures.

Another embodiment of the apparatuses, systems and methods disclosed will include robotic technology. In general, robotic surgery involves surgical procedures using robotic systems. In the case of robotically-assisted minimally-invasive surgery, instead of directly moving the instruments by hand while holding onto the instrument, the surgeon will typically use one of two methods to control the instruments: direct telemanipulation, or computer control, which allows the surgeon to perform the normal movements associated with the surgery. The robotic arms carry out those movements using end-effectors and manipulators to perform the actual surgery.

In computer-controlled systems, the surgeon uses a computer to control the robotic arms and its end-effectors, though these systems can also still use telemanipulators for their input. One advantage of using the computerized method is that the surgeon does not have to be present, leading to the possibility for remote surgery.

The apparatuses, systems and methods disclosed will provide to the neurosurgeon all of the features and functions needed for the entire surgical procedure in a fully integrated package that will allow for efficient and effective remove of the clot without the need for a craniotomy—from initial assessment of the clot, its size, location and status, to planning of the surgical procedure (from scalp incision to trajectory and depth of insertion of the cannula, to controlled addition and removal of temperature-controlled fluid to the clot site, to precise and controlled removal of the clot, to confirmation of desired removal, to cauterization—and all done with real time visualization on adjacent video monitors that will allow the surgeon to “see” inside the brain.

Other advantageous aspects and benefits of the apparatuses, systems and methods disclosed will be apparent to those skilled in the art. Nothing stated above is intended to limit the scope of the protection provided for the inventive concepts which are disclosed herein. Herein, the words “hemorrhage,” “hematoma,” “clot” and the acronym ICH are used interchangeably to refer to blood, whether clotted or not, that is present in an unwanted part of the brain. The word “tumor” is used in its ordinary and customary way. The system and methods herein described for removal of a clot can also be utilized for removal of brain tumors, although it will be apparent to those skilled in the art that tumor-removal-specific tools may also be utilized in addition to those described below.

### DESCRIPTION OF THE FIGURES

FIG. 1 is a graphic representation of the patient laying on the operating table, showing the patient's head, the approximate location of the brain within the patient's skull, and a representation of a blot clot within the brain.

FIG. 2 is a graphic representation of one embodiment of the system's primary components shown in conjunction with the patient on the operating table.

FIG. 3 is a graphic representation as in FIGS. 1 and 2, but with an isolated view of three of the systems components—an alignment mount (attached to the surgical table) to be used to precisely guide the cannula for insertion and the neuronavigation alignment probe which will also be inserted into the alignment mount's bushing. This Figure essentially depicts initial set-up of certain components in conjunction with the patient positioned for surgery.

FIG. 4 is a graphic representation as in the FIG. 3, showing the neuronavigation tool inserted into the alignment mounts bushing. The arrows depict the rotational movement to determine the desired access pathway through the patient's skull into the clot.

FIG. 5 is similar to the preceding Figures, but in this Figure the craniotome (a cranial drill) used to drill an access port (or burr hole) for the system's components that will be inserted into the patient's brain. Here, craniotome is shown positioned for insertion into the bushing of the alignment tool and then for precisely controlled drilling of the access port in the patient's skull.

FIG. 6 is similar to FIG. 5, but in this Figure the access port or burr hole has been drilled, and the craniotome removed. The system's obturator (which is used to create a pathway to the clot (or tumor) through the folds of the brain without damaging the brain) is positioned for insertion first into the alignment tool's bushing, and then through the access port in the patient's skull, and then into the depicted blood clot (or tumor).

FIG. 7 is similar to FIG. 6, but shows the obturator (and in this embodiment, a surrounding peel-away sheath now fully inserted into the brain such that the tip of the obturator is located within or immediately adjacent to the to-be-removed clot or tumor.

FIG. 8 is similar to preceding Figures, but in this Figure the obturator has been removed, leaving the peel-away sheath in-situ within the brain to maintain the pathway to the close (or tumor) after the obturator has been removed.

FIG. 9 is similar to the preceding Figures, showing the system's cannula (in this embodiment, having multiple lumens or channels that run the entire length of the cannula) about to be inserted into the patient's through the peel-away sheath that maintained the pathway to the clot after removal of the obturator.

FIG. 10 is similar to FIG. 9, showing the multi-lumen or channel cannula now fully inserted into the peel-away sheath and into position within the patient's brain.

FIG. 11 is similar to FIG. 10, showing the distal end of the blot removing device now positioned for insertion through the cannula (which has remained within the peel-away sheath) and into position within the patient's brain to begin clot removal. Of course, in actual practice and use of the described system and methods, these various components that are inserted into the patient's brain are being guided either manually by the surgeon, or robotically using know techniques (neither of which are shown in the Figures).

FIG. 12 is similar to FIG. 11, showing that the clot removing the distal end of the clot removing device has been fully inserted through the cannula and is now positioned within the clot in the patient's brain to begin clot removal.

FIG. 13 is similar to the preceding Figures, showing that the clot removal operation has been completed and the clot removal device has now been extracted from the patient's brain. It is noted that the cannula within the peel-away sheath remain in-situ.

FIG. 14 is similar to the preceding Figures, but shows that that after the clot removal device has been extracted, other tools can be inserted through the cannula into the patient's brain, for example cautery, another scope, or an ultrasound probe can be inserted through one of the functional channels in the cannula into the patient's brain. It should also be noted that because a preferred embodiment of the cannula will have multiple channels or lumens, other such devices could be used before the clot removal device is extracted.

FIG. 15 is similar to preceding Figures, showing the situation in which all intracranial procedures have been completed, and the cannula and peel-away sheath have been removed.

FIG. 16 depicts the peel-away sheath, the obturator and the neuronavigation positional marker.

FIG. 17a and FIG. 17b show two perspective views of the multi-lumen or -channel cannula which will be inserted into to the patient's skull. The neuronavigation position markers are also shown attached to the cannula

FIGS. 18a and 18b show two different views of an alternate embodiment in which the obturator and the cannula are combined into a single component. Here, the component has a soft tip that is used during the obturating step, and behind the soft tip, a side exit port through which the tip of the clot removal device can be extended once the side port of the cannula is within the to-be-removed clot (or tumor). In FIG. 18a the distal tip of the clot removal device is shown in a retracted position within the cannula. In FIG. 18b, the distal end of the clot removal device is show extended through the exit port in the cannula. As shown here, the distal end of the clot removal device can be articulated to allow it to protrude though the cannula's exit port.

FIG. 19 depicts the combination obturator/cannula with inserted clot-removal tool in position to be inserted into the patient's skull.

FIGS. 20a and 20b depict the distal ends of the combined obturator/cannula/clot-removal device inserted into the clot within the patient's brain. The distal tip of the clot-removal tool is extended through the exit port so that the tip is outside and extending laterally from the cannula. The rotational arrow in FIG. 20b depicts how clot removal device can be rotated so that its extended tip can rotate within the clot for efficient and effective breaking up and removal of the clot.

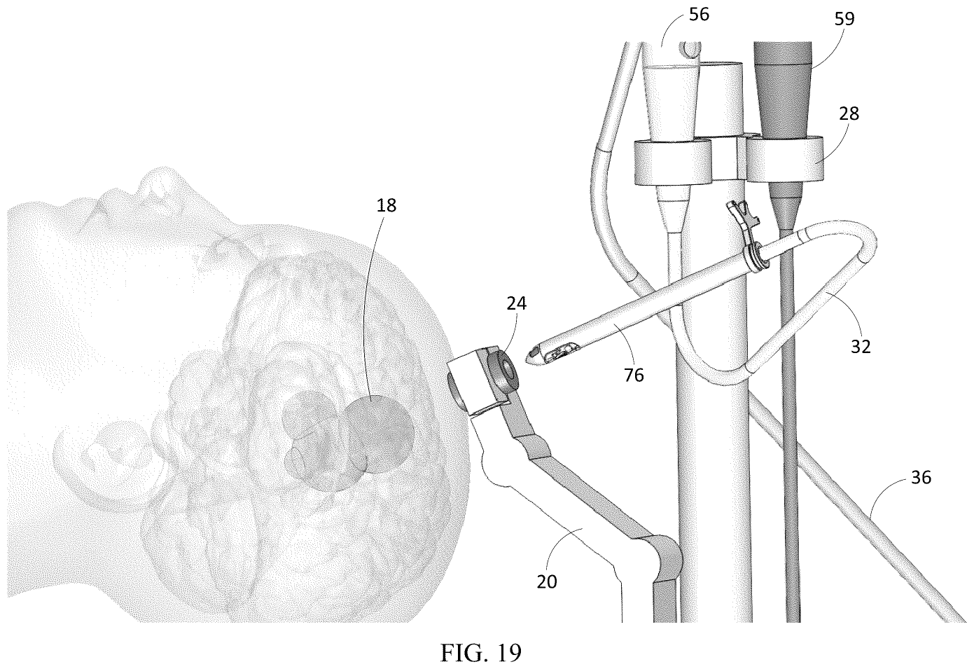

FIGS. 19a and 19b the clot-removal tool in isolation (that is, not inserted into the cannula. In FIG. 19a, the distal tip of the tool is articulated, and in FIG. 19b it is not. The neuronavigation position marker is shown in position on the tool.

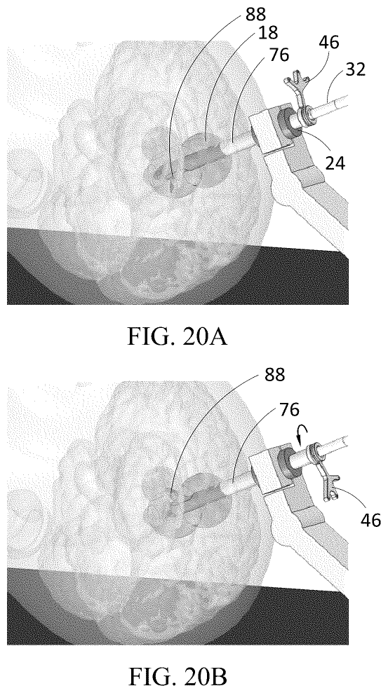

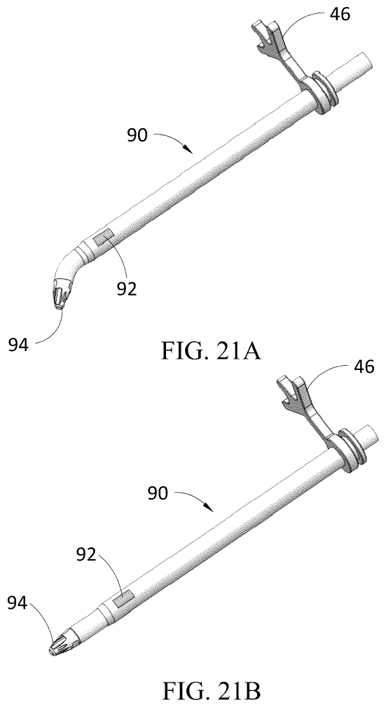

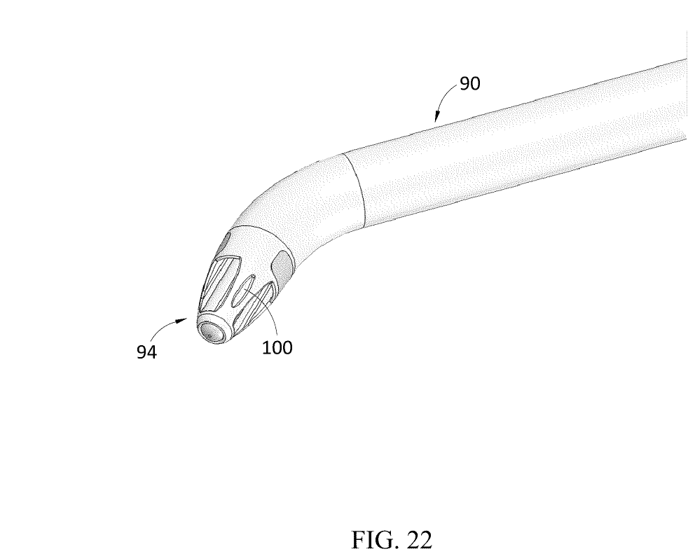

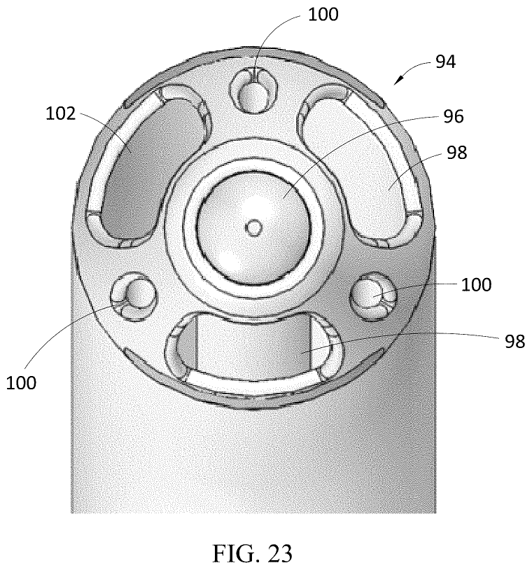

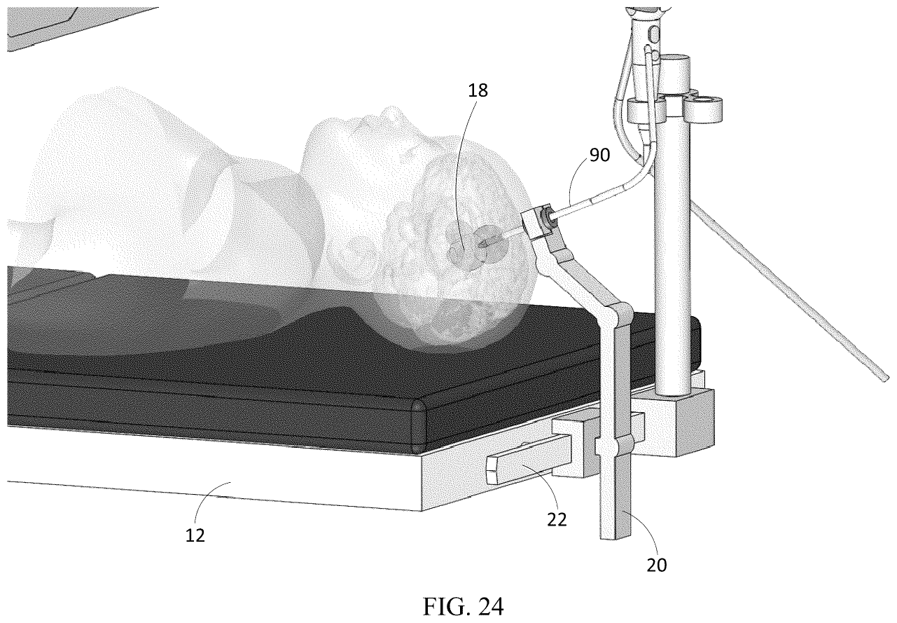

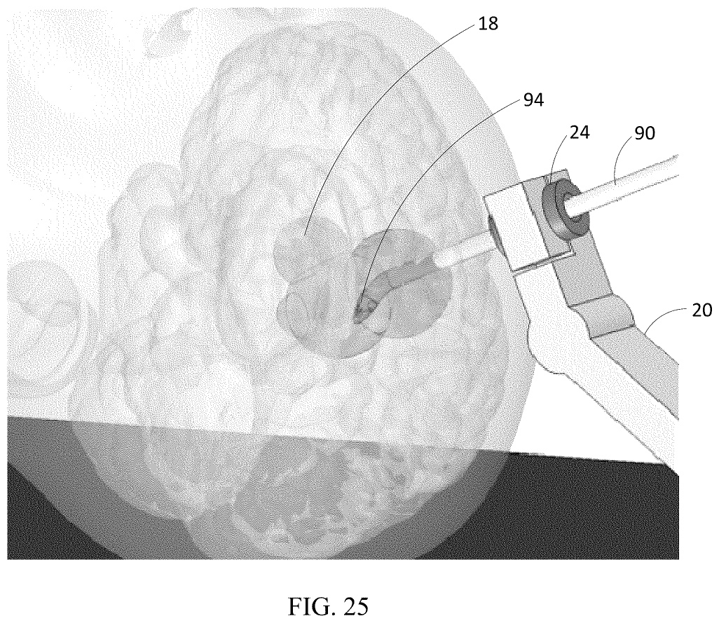

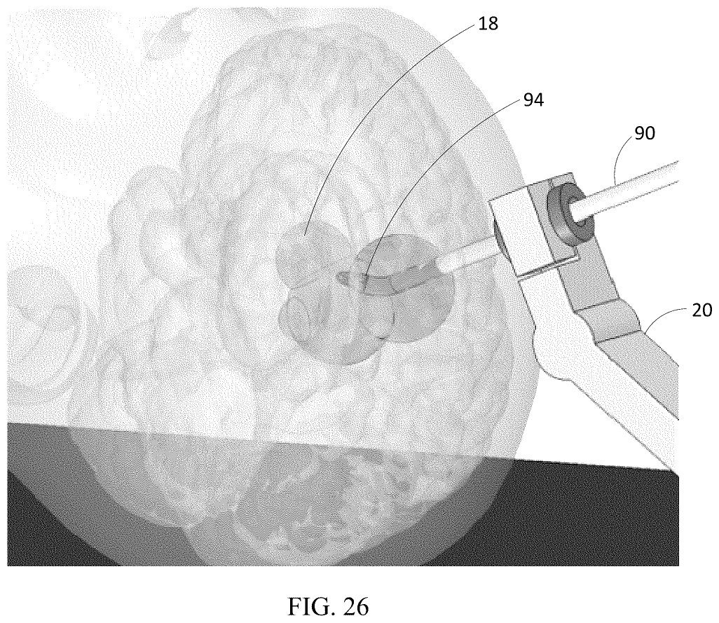

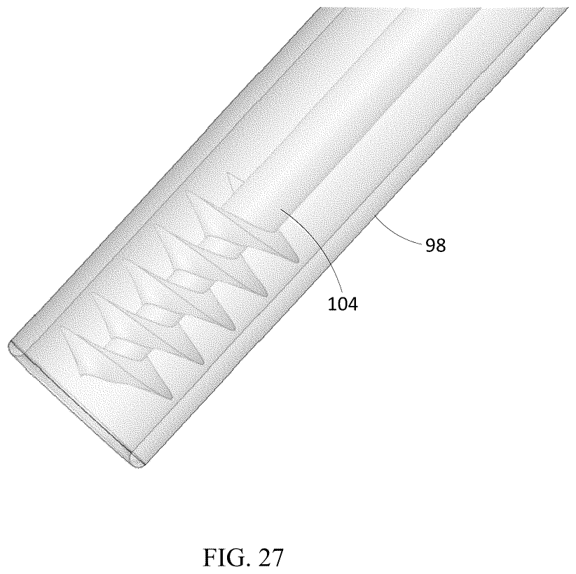

FIGS. 20a and 20b show the combined cannula with the obturation tip in place within the to-be-removed clot and position to begin the removal step, and with distal end of the clot removal device in the extended position outside and lateral positioned relative to the cannula. FIG. 20a shows the tip of the clot removal tool pointed posteriorly, and in FIG. 20b it is pointed anteriorly. This rotational movement is created by rotation the cannula as shown by the directional arrow in FIG. 20b.

FIGS. 21a and 21b show another preferred embodiment of the combined obturator/cannula device which would be inserted into the patient's brain through the peel-away sheath. In FIG. 21a, the distal tip is shown in an articulated position (which would be used after the tip is inserted into the to-be-removed clot); and in FIG. 21b it is shown in a non-articulated position (which would be used initially when the device is first inserted into the patient's brain).

FIG. 22 is a close-up view of the distal end of the clot removal device in an articulated state, and shows the various access ports that can be included in the clot removal device.

FIG. 23 is a close-up view of the distal end of the clot-removal device looking directly at the very end of the device, which depicts the various components that can be included, such as a scope, a multi-sensor location, and irrigation channels and evacuation channels, for examples.

FIG. 24 shows a situation in which the clot-removal tool depicted in FIGS. 22 and 23 is inserted into the clot through the alignment bushing, and with the distal end of the device not yet articulated.

FIGS. 25 and 26 show the clot-removal tool depicted in FIGS. 22 and 23 inserted into the clot through the peel-away sheath and the alignment bushing, and with the distal tip of the device not yet articulated. The rotational arrow in FIG. 26 shows that the tool has been rotated within the clot between that shown in FIG. 25.

FIG. 27 shows an alternative embodiment of the distal end of the clot removing device in which a helical auger-type, rotatable element is incorporated into the end of the clot removing device to facilitate the removal of the clot material.

These and other aspects of the disclosed system and method will be further described below, and are not limited to those depicted in the Figures.

### DETAILED DESCRIPTION OF THE PREFERRED EMBODIMENTS

In a typical ICH situation, the patient symptoms once the aneurysm has ruptured will be obvious and extreme, such that immediate transfer to the hospital will occur. The patient will typically be admitted via the hospital's emergency room. The immediate goal will be to stabilize the patient as much as possible. The patient is then sent for imaging, in which an initial CT scan of the head, and an angiogram of the head and neck regions. A neurosurgeon will be consulted and an MRI will also be taken in most cases. The imaging results will show the size and location of the ICH, and will provide the baseline data for treatment, and for use by the neurosurgeon using the apparatuses, systems and methods herein disclosed. The preferred system will work with CT or MRI system or portable CT (O-Arm), with the advantage of the O-Arm being that it can be done real time in the Operating Room, and can include augmented visualization. The patient's age, and the location, size and status of the ICH will dictate treatment, including if surgery is required to remove the clot.

In the preferred embodiment, the system will include integrated neuronavigation, which will initially be utilized for planning the location, orientation and size of the skull skin incision, the size and location of the access hole to be drilled in the skull, and the trajectory and depth for insertion of the cannula. Once these parameters have been determined, the surgery can begin using the preferred embodiments of the disclosed apparatuses, systems and methods, which include hardware and software.

The hardware components can include an integrated video screen or screens for initial visualization of the clot in-situ via ultrasound or other similar means; an integrated and highly controlled vacuum pump for fluid aspiration, coordinated with an integrated pump system for highly and accurate controlled fluid delivery to the clot site so that fluid being removed is equally offset by the volume of fluid being added to avoid undesired fluid pressure differential in the brain. The apparatus can also include an integrated heat-exchange system to allow cooling and temperature control of the fluids to be delivered into the brain.

The hardware will also preferably include an integrated fluid oxygenation and oxygen content measurement capability for the fluid being delivered to the brain in either PO2 or SO2. The preferred system will also monitor and control the pH of the fluid being added. An integrated sensing capability will also be preferably included, which will sense, monitor and measure such parameters as pressure, temperature, oxygen levels, fluid delivery and aspiration volumes, vacuum level. A Mayfield three pin head holder (or similar device) will be used to hold the patient's skull in a motionless position, and also for attachment of certain of the hardware components. The hardware will also preferably include integrated control and safety mechanisms to ensure proper operation.

The hardware components will also include single-use disposable items as well. A very important component is a dual lumen or multi-lumen cannula that can be 4 mm-12 mm in diameter and 6 cm to 12 cm in length. The lumens provided with the system can be various diameters. The working-end tip of the cannula can be rigid or flexible, and can also have steerable tip with varying lengths. The preferred cannula will incorporate sensors for pressure, temperature and oxygen levels, and the sensors can be at tip or along entire body of cannula in varying increments from each other (i.e. 1-2 cm apart).

The hardware components will also preferably include an ultrasonic probe for visualization similar to Intravascular Ultrasound (IVUS) technology. The probe can be mounted as fixed probe at the tip of cannula or part of flexible tip. The probe can also be introduced through a large lumen in cannula that can be pushed through the length of the cannula and beyond cannula tip into hematoma via a guidewire type of system. Once the probe is inserted into the cannula to a point where it extends beyond tip of cannula, the cannula lumen can be used for other purpose such as fluid delivery or aspiration of clot or fluid; can also be used for other purposes as required. The ultrasonic probe is connected to a video screen to display and show details of the hematoma, such as visualization of residual contours of hematoma. The objective here is to “see” the geometry, size, volume, density of clot (i.e., whether it is entirely or mostly, fluid or of a more solid consistency. The details are important for the surgeon to determine the method and tool by which the hematoma will be extracted. The probe will also be able to determine and differentiate the cellular make-up between blood clot and brain tissue.

The preferred apparatus, system and method will include the ability to circulate continuous compatible hyperosmotic hypothermic 23% NaCl solution (or other desired solutions) through the cannula into the clot site. As mentioned, the fluid delivery and aspiration will needs to be controlled, as fluid delivery will not be allowed by the system exceed fluid aspiration. This will include pressure monitoring integrated to constantly measure the fluid pressure within hematoma. Safety systems will be preferably included to restrict fluid volume delivery not being allowed to exceed fluid volume aspiration. Fluid oxygenation capability will also be preferably integrated into the system. Controlled and monitored, cooling of the fluid may be desirable to offset and dissipate the heat that may be generated at the clot site due to, for example, ultrasound removal of the clot. In addition, the introduction of cooled fluids can help to reduce swelling in the surrounding brain tissue and provide neuro-protection.

Various clot breakup mechanisms will be integrated into system. The mechanisms will need to be able to dissolve acute and subacute blood clots. Options for the mechanisms include: ultrasonic—mechanism delivered though a lumen in cannula or integrated fixed into cannula tip to break up clot (this will calibrated, controlled and monitored to differentiate between clot blood cells and brain cells, so that there is controlled and precise aspiration of the clot with only minimal damages to adjacent brain cells. The preferred system will also provide for continuous or intermittent fluid delivery to the clot site to dilute clot and also to cool ultrasonic clot break-up mechanism if heat is generated. Fluid delivery and aspiration will be controlled and will include integrated safety control that does not allow fluid delivery to be greater than aspiration. The preferred system will continuously measure, monitor and report both volume and pressure of the fluid at the clot site.

Other mechanical clot break-up systems can be included. For example, high speed roto-rooter type or burr tip technology that has cell differentiation recognition capability to assess blood cells (clot material) vs brain cells; or an Archimedes screw type of mechanism that can pull as well as potential break up clot within cannula; or a brush type system—a spiraled brush system along a catheter that spins and pulls clot. Brush can be soft or metallic to break or chop up clot.

The software that is integrated into the preferred system interfaces with a Neuronavigation system and CT/MRI imaging to provide instantaneous and precise feedback on positioning of the cannula as it is being navigated through the access hole in the skull, into the brain, then into the clot, and includes an indication of the cannula's location within clot. The software will also provide instantaneous feedback on the amount of clot being removed, as that is compared against the baseline data that was captured from the initial imaging (CT and MRI) of the patient's skull.

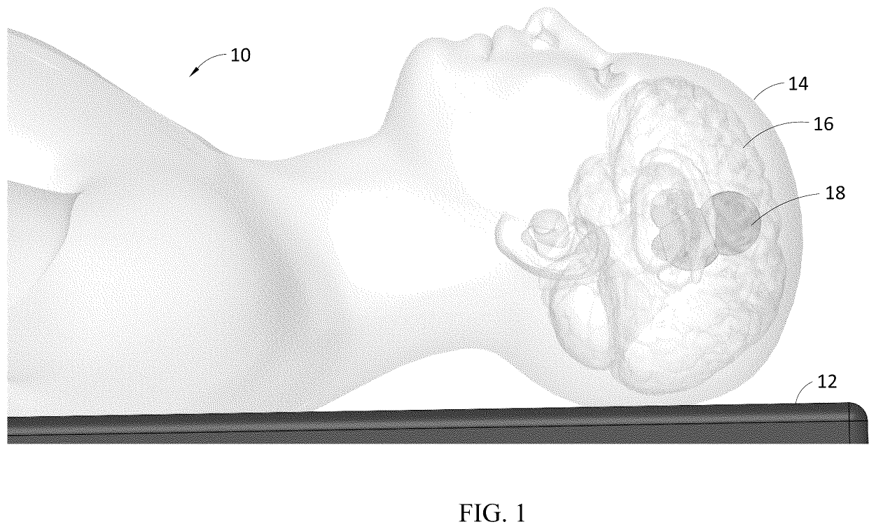

Referring now to FIG. 1, a patient 10 is shown lying on a typical surgical table 12, having been previously prepped for brain surgery. In a typical brain surgery, the patient's head would normally be immobilized against the table 12 by means of a strap or other similar device (not shown). This patient's skull 14 encases the patient's brain 16, in which resides the to-be-removed clot 18.

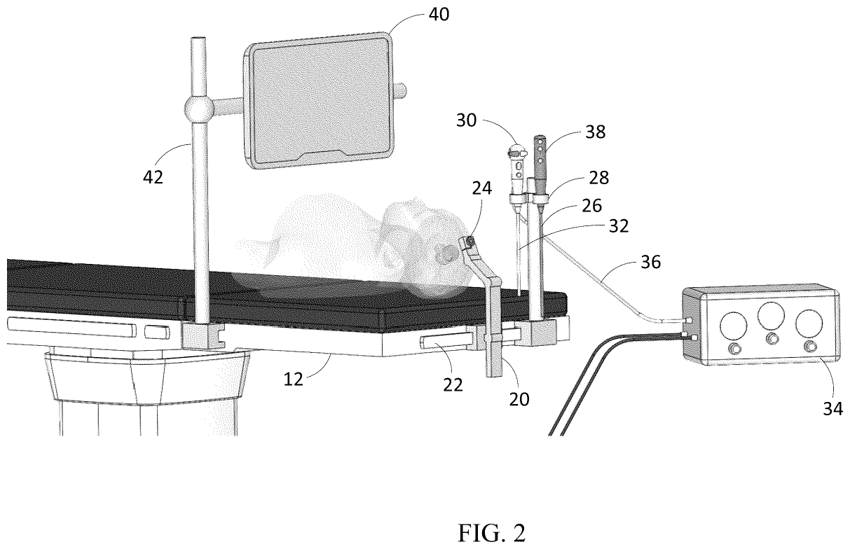

FIG. 2 shows the same prepped-for-surgery patient 10 prone on the table 12 along with certain of the other components of the overall system. The alignment mount 20 is adjustably attached to the table 12 by means of bar 22. The alignment mount 20 will be used for controlled and exact insertion and use of the main tools to access and then remove the clot; the alignment bushing 24 that is located in and locks into the upper end of the mount 20, and through which the various tools will be inserted for precise location and angle-of-insertion control relative to the brain 16 of patient 10, and the location of the clot 18 within the brain.

A multi-handle rack 26 is movably attached to bar 22. The upper portion of the rack 26 is preferably outfitted with open collars 28 into which the handles of the various tools that the surgeon will use or may want to use can be located in easy reach by the surgeon. For example, as shown in this FIG. 2, the multi-handle rack 26 is holding the handle portion 30 of the intracranial clot-removal device 32.

The clot removal device 32 is shown in FIG. 2 in which the handle portion 30 of that device is residing within one the rack collars 28. The clot removal device 32 is connected to the control box 34 seen in FIG. 2 via connection tube 36. The control box 34 can be of any standard size, shape, configuration and functionality to provide and control the various devices that are being used with the system, such as device 38, which will also be held in one of the collars 28 in rack 26. The control box 34 in a preferred embodiment will provided for adjustable suction pressure, irrigation pressure, irrigation temperature ambient fluid pressure within the brain, and the controlled inflow and outflow of fluid so as to maintain a proper fluid pressure within the patient's brain. The control box 34 can also monitor the amount of clot material removed. It can also provide for other devices and functions such as O2 sensing, for example. The system will also include a monitor 40 which will be attached to a monitor mount 42 that is attached in this embodiment to the surgical table 12, and can be adjusted so that the monitor 40 can located as desired by the surgeon. It is preferred that the monitor (or monitors as the system can provide for multiple monitors) be locates in such a way that the surgeon will have easy sight-line access so as to eliminate the need for the surgeon to move his or her head in order to view the monitor 40, thereby eliminating unwanted movement that might be transmitted to the working end of the devices once inserted into the patient's brain 16.

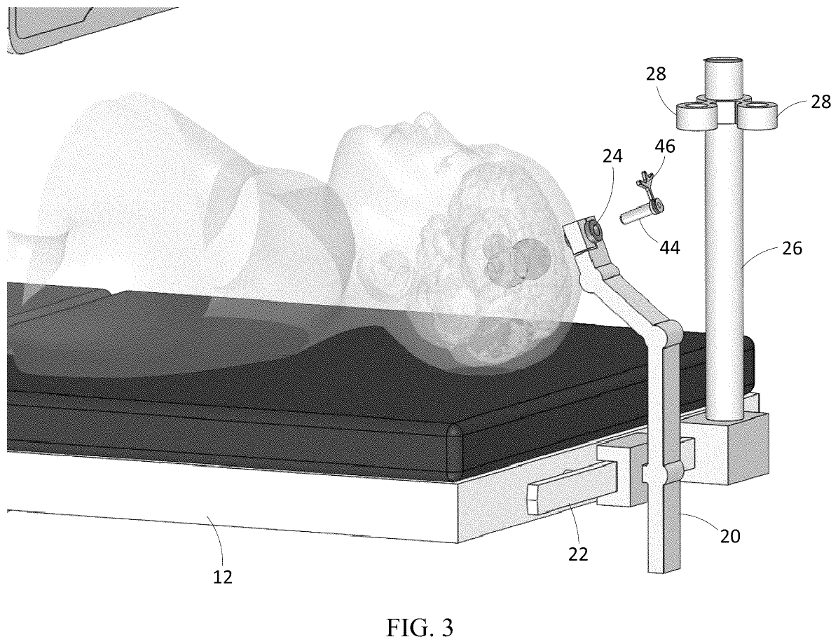

The initial set up of the system as shown in FIG. 3 includes the neuronavigation alignment probe 44 and its affixed position markers 46. As is well known to those of skill in the art, neuronavigation is the set of computer-assisted technologies used by neurosurgeons to guide or “navigate” within the confines of the skull during surgery. (See, e.g., https://en.wikipedia.org/wiki/Neuronavigation). There are many such suitable systems available commercially, and the apparatus, system and methods herein described and claimed can be used with them. The position markers 46 are required by the standard and known neuronavigation software to determine the optimum path for insertion of the obturator device into the patient's brain to gain access to the clot. The probe 44 is inserted into the alignment bushing 24.

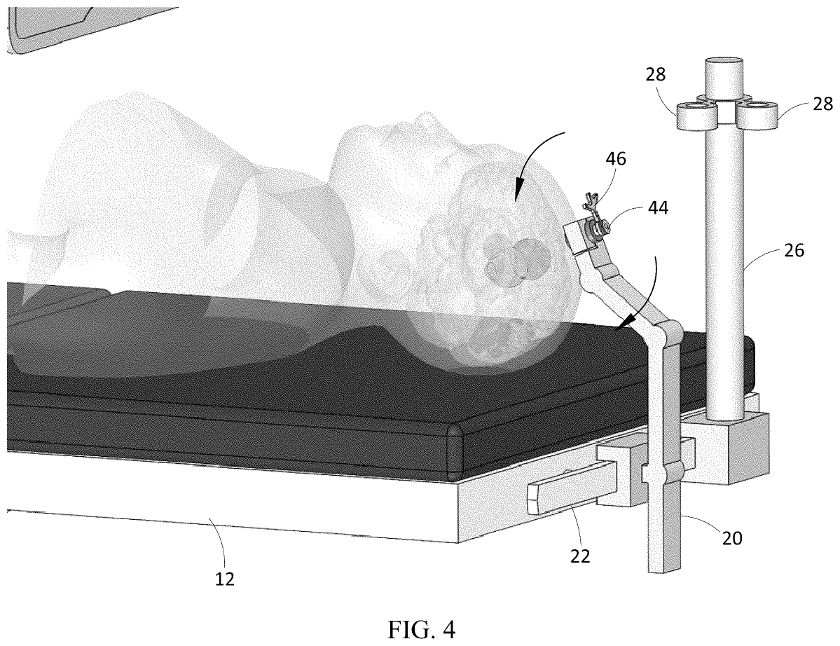

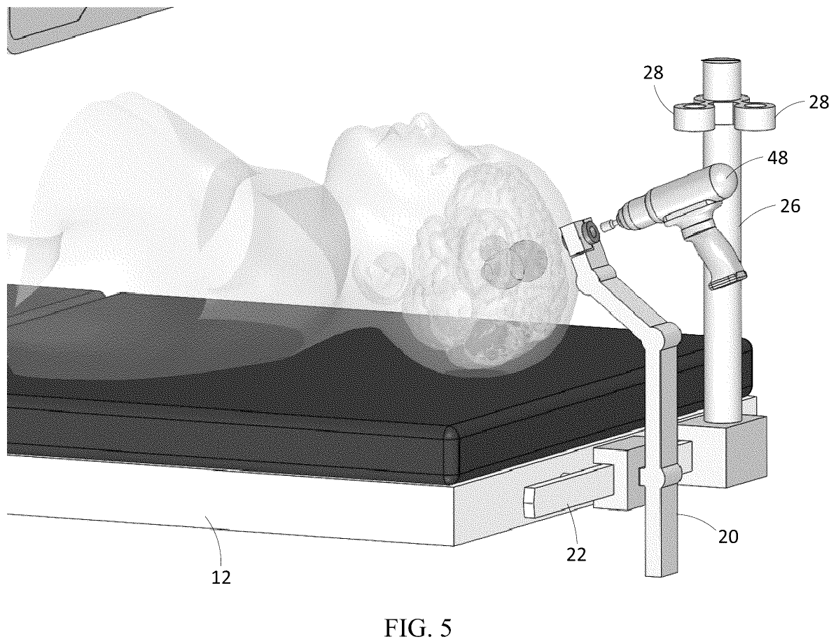

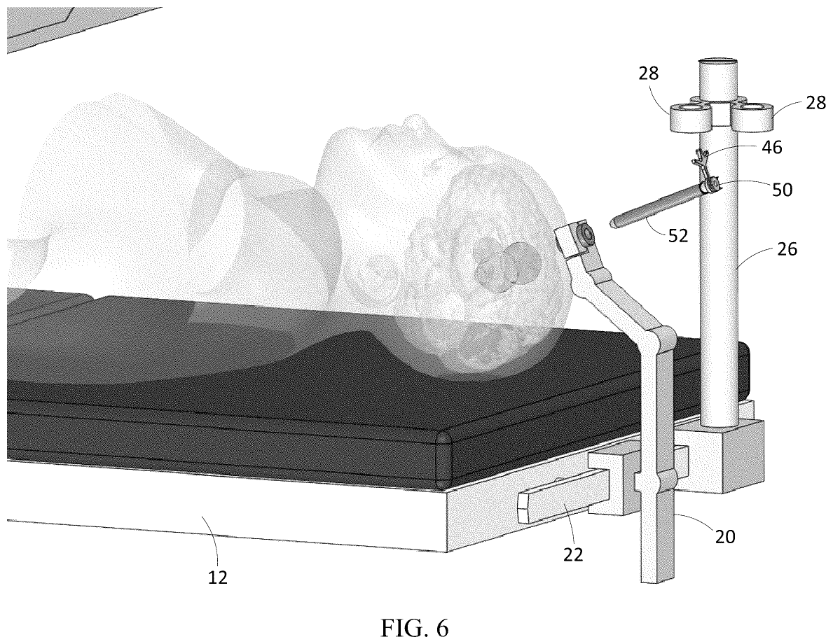

As best seen in FIGS. 4 and 5, once the neuronavigation alignment probe 44 has been inserted into the alignment bushing 24, the adjustable mount 20 is rotatable along multiple axis to achieve the optimal access pathway as determined using the neuronavigation software. Once that optimal pathway is determined and the adjustable mount 20 is locked into place, the next step in the procedure is to drill a small access port or burr hole into the patient's skull using a craniotome 48.

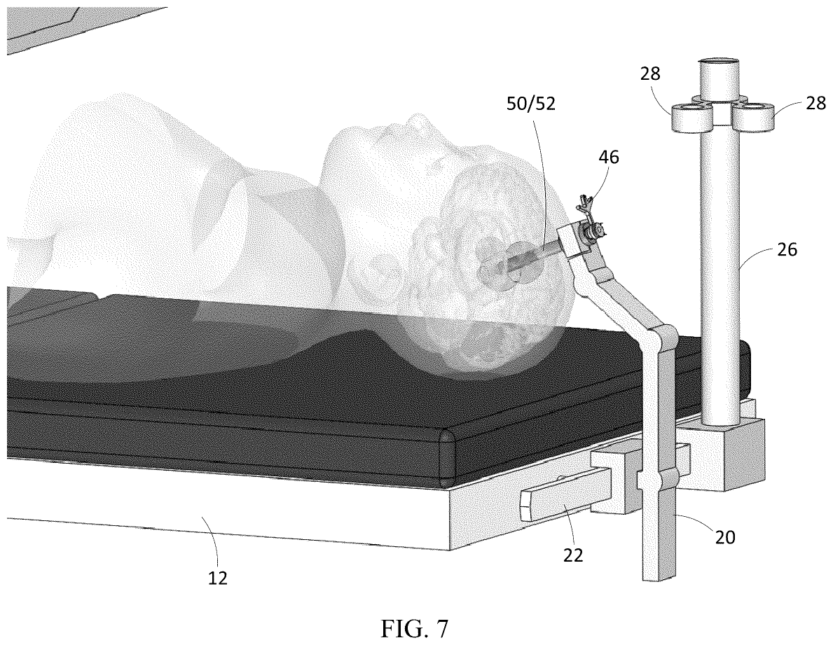

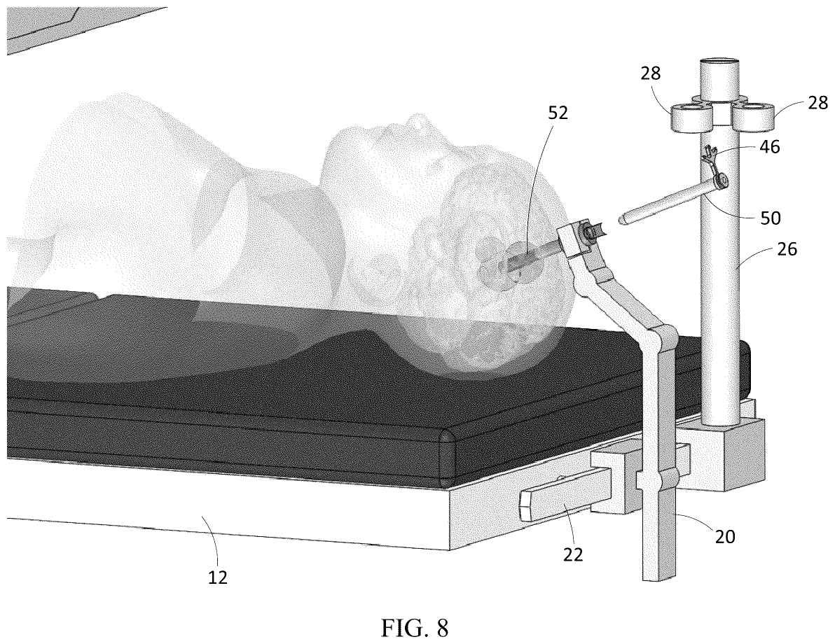

Once the burr hole (not shown) has been created, the next step is to create an access pathway within the patient's brain 16 to the clot to be removed, and to do so without injuring healthy brain tissue. An obturator 50 is used for this procedure. In a preferred embodiment, the obturator 50 will first be inserted into a peel-away sheath 52 as best seen in FIG. 6. The obturator 50 and sheath 52 are then inserted through the alignment bushing 24 on the alignment mount 20 and is then inserted through the burr hole into the patient's brain until the tip of the obturator 50 reach the clot 18 to be removed, as shown in FIG. 7. The peel-away sheath 52 will remain stationary within the patient's brain even after the obturator 50 is removed, and will provide for a reusable passageway to the clot location for other devices as hereinafter described. This is best seen in FIG. 8. It is noted that the surgeon can leave the sheath 52 inserted throughout the entire procedure, or remove it, at his or her choice.

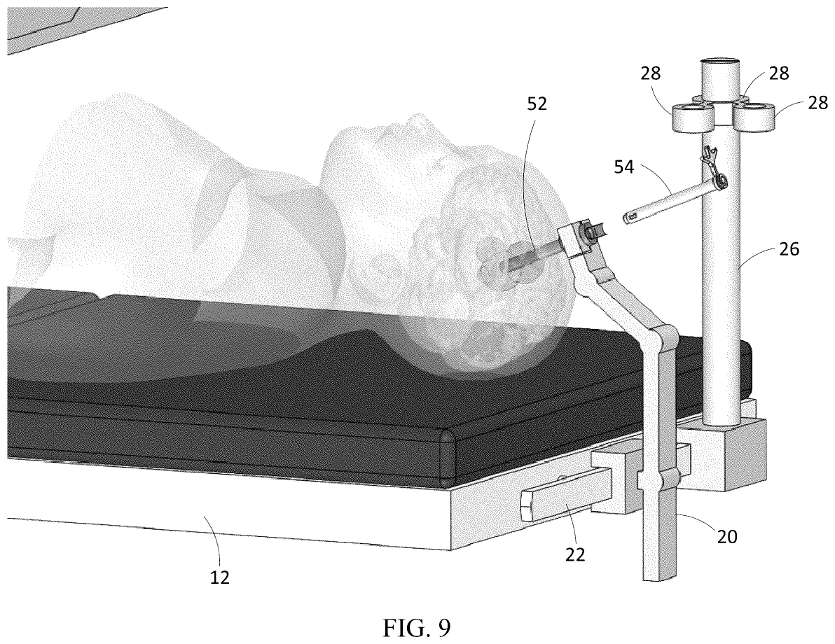

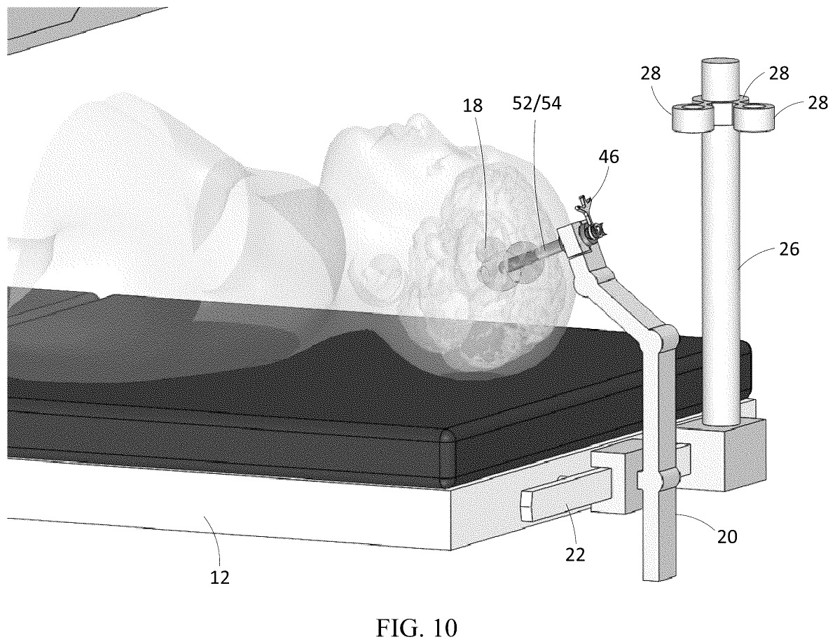

At this point in the procedure, a preferred embodiment of the cannula 54 is readied for insertion through the peel-away sheath 52 into the patient's brain 16 and down to and into the to-be-removed blood clot 18. As best seen in FIG. 9, the cannula 54 is sized and shaped so that it fits snugly within the peel-away sheath 52 but can be slid easily within it. The completed procedure whereby the cannula 54 has been fully inserted into the peel-away sheath 52 is seen in FIG. 10. As will be discussed in more detail below with reference to other Figures, a preferred cannula 54 will have a number of elements and features. At this point in the procedure, an access pathway has been created to the clot 18 which will be used for the various tools and devices that the surgeon will need to insert into the patient's brain in order to remove the clot.

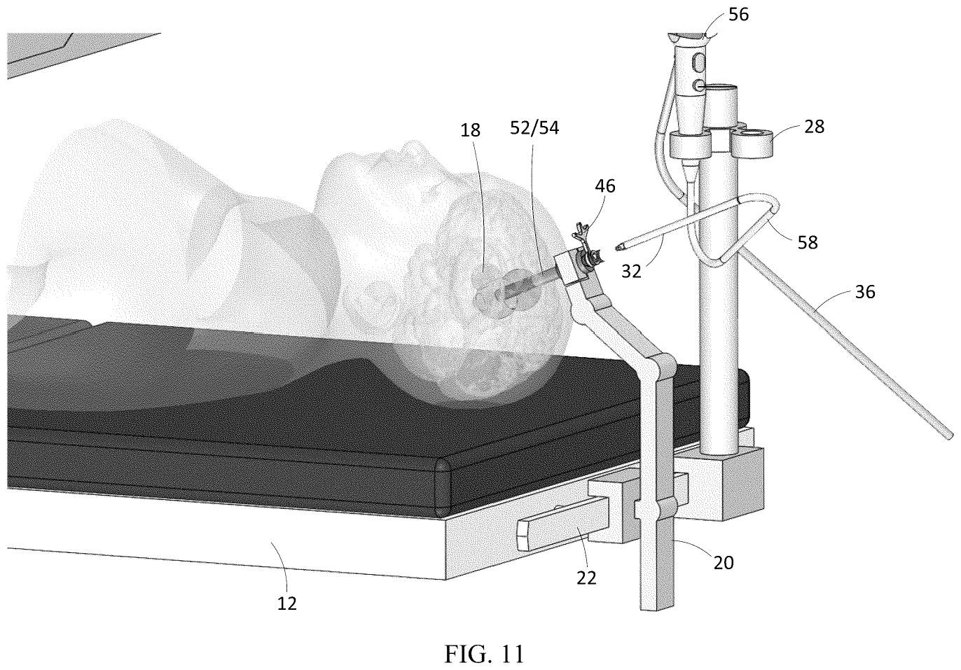

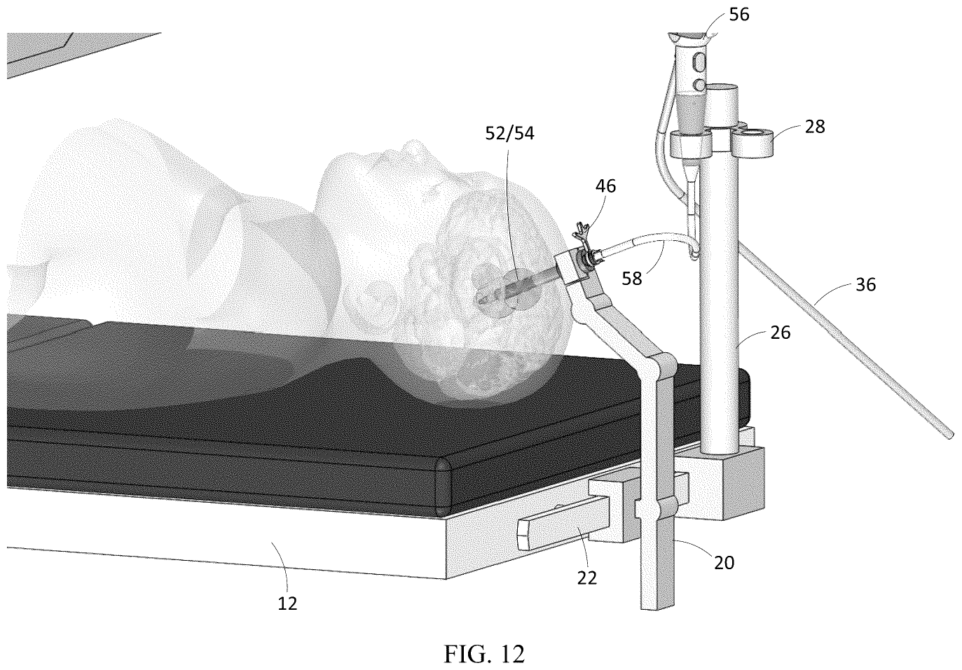

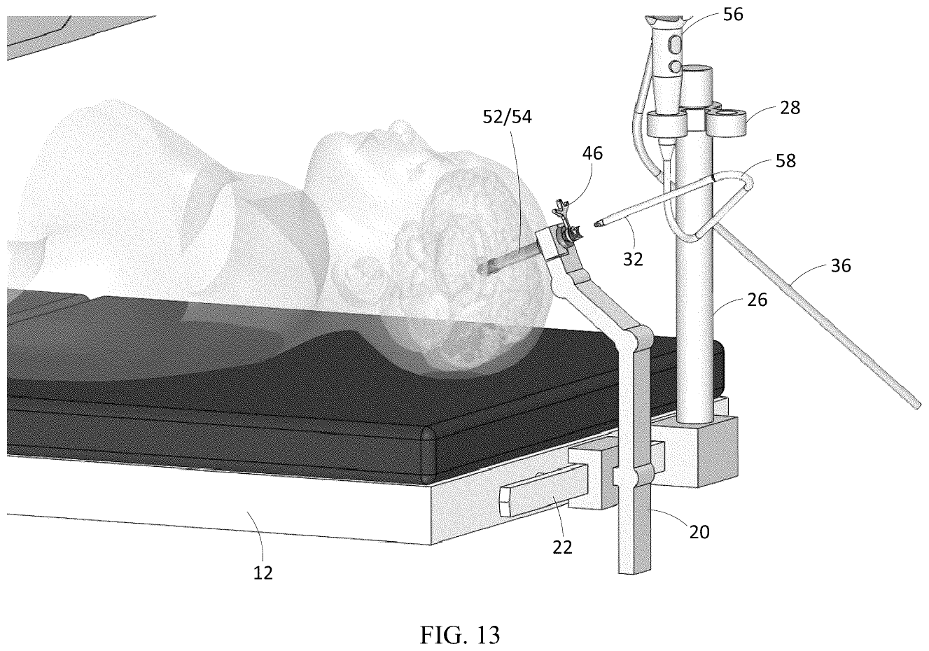

The next step in the procedure involves inserting the distal end of the clot removal device 32 into the to-be-removed clot 18. As described above, there has now been created an access pathway to the clot 18, and that pathway is through, the cannula 54 that resides within the peel-away sheath 52, which is held in position by the alignment bushing 24 that his held in position within the alignment mount 20. As best seen in FIG. 11, a preferred clot removing device 32 will have a handle 56 to which a tube 58 is attached, and at the distal end of the tube is attached the device that will accomplish the clot removal. As shown in FIG. 12, the clot removal device 32 has been inserted into the clot 18 but no portion of the clot has yet been removed. Typical clot sizes are 50 to 70 cc in volume. What is shown in FIG. 11 is an approximately 50 cc clot. FIG. 13 depicts the clot size after the removal procedure is completed, which is now approximately 5 cc. It is typical in such operations that the entire clot is not removed, otherwise hemorrhaging at the clot site may resume. Once the desired amount of clot material has been removed, the clot removal device 32 is removed from the cannula 54 as is depicted in FIG. 13. It is noted that the cannula 54 remains in place.

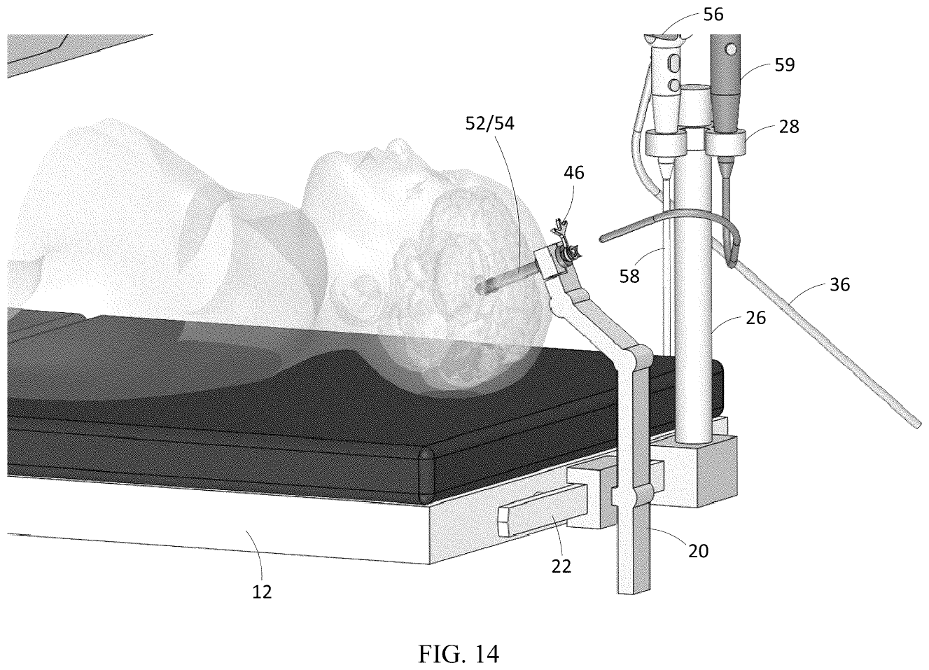

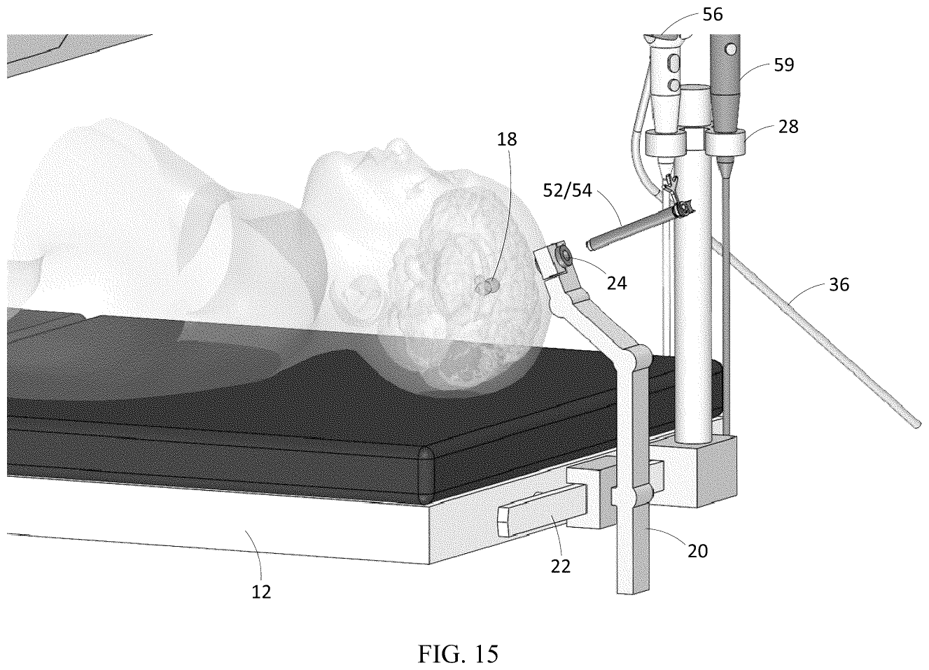

Because the cannula 54 has remained place, other devices can be used by the surgeon as desired, and inserted through the cannula 54 as shown in FIG. 14. Such other devices could include (and not limited to) a visual scope, an ultrasound probe, device to cauterize the wound site within the brain if hemorrhaging continues. These other devices 59 can be inserted through a main channel within the cannula 54. In addition, because the cannula 54 can include multiple channels (as will be described in more detail below), one or more of these other devices can be inserted through the cannula 54 and used simultaneously with the clot removal device 32. In other words, the surgeon will have the option of using such other tools before or after the clot removal device 32 is removed. Once all procedures within the patient's brain are concluded, the cannula 54 and peel-away sheath are removed, and the burr hole dressed and sealed to prevent leakage of any cerebral/spinal fluid. Sealing around the peel-away sheath at the burr hole site during the procedure is also preferred to prevent such leakage of the cerebral/spinal fluid.

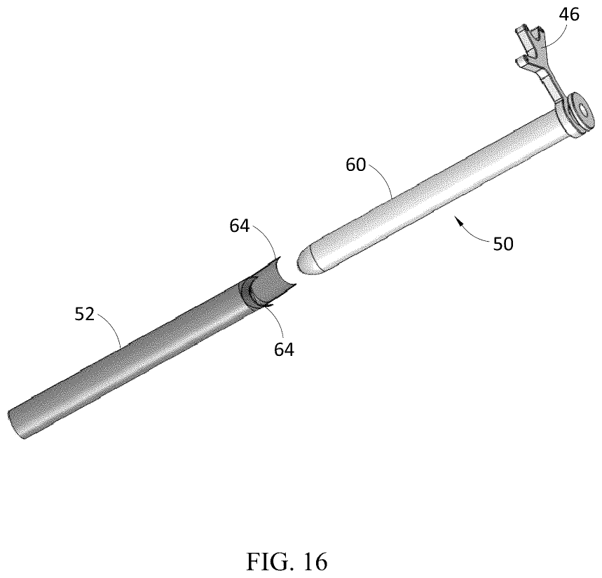

One of the preferred embodiments of the obturator 50 is seen in FIG. 16. As shown there, it has a body 60 of consistent exterior radius that mates with the interior radius of the body portion 62 of the peel-away sheath 52. The sheath 52 can have a pair of flaps 64 at the proximal end of the sheath for easy peeling away. As shown here, the obturator 50 is equipped with the neuronavigation position markers 46. In the embodiment of the obturator 50 shown here, there is a single channel 64 that extends the entire length of the obturator 50 that will accommodate insertion of a scope (not shown).

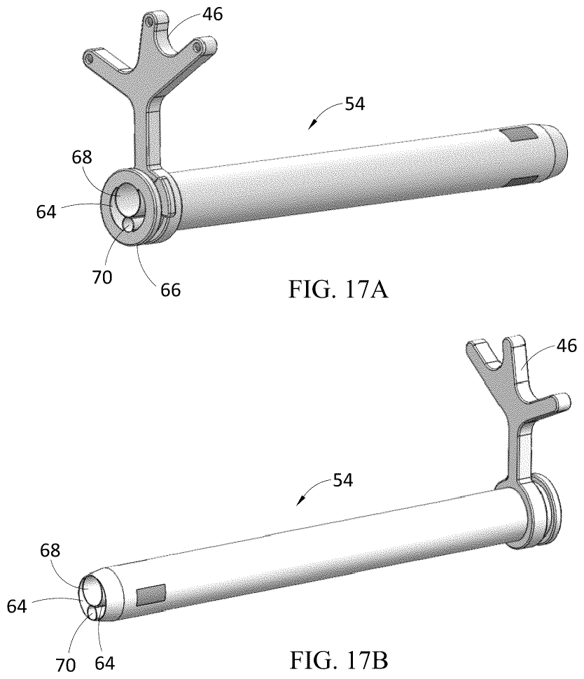

Preferred alternative embodiments of the cannula 54 are described below. FIGS. 17a and 17b depict one of the preferred embodiments in perspective views from two different angles. In FIG. 17a, the proximal portion 66 of the cannula 54 is visible and shows that this embodiment of the cannula 54 has a main channel 68 that is preferred intended for use with the clot removal device 32; a secondary channel 70 that can be used for example, with the other devices described above, such as a visual scope, ultrasound or cautery; and two side channels 64 that can used for other purposes that the surgeon may desire. As shown in FIG. 17b, each of these channels extends the entire length of the cannula 54. In this embodiment, the cannula 54 has a pair of multi-sensor location 72 and 74 which can be used for sensing such things as fluid pressure in-situ, O2 levels, temperature, etc. As also depicted here, this embodiment of the cannula 54 includes the neuronavigation position markers 46 fixedly attached at the proximal end of the cannula 54.

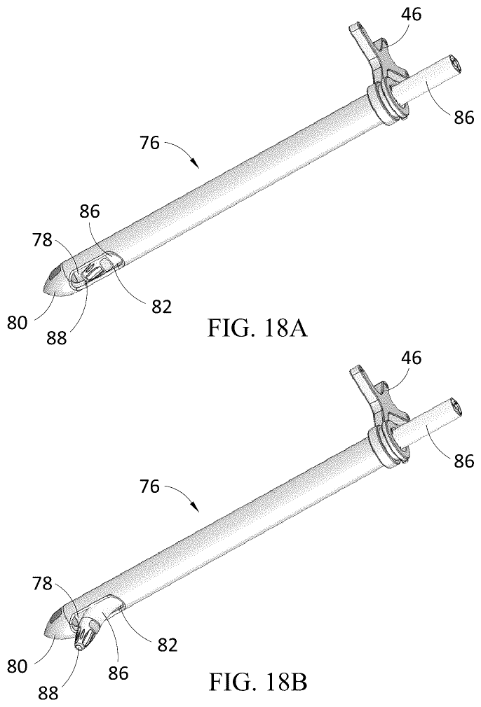

An alternative embodiment is shown in FIGS. 18a and 18b, in which an obturator and cannula are combined into a single device 76. This device 76 preferably has a single channel 78 into which another embodiment of clot removal device 34 can be inserted. This embodiment of the combined obturator/cannula device 76 also includes the fixedly attached neuronavigation position markers 46 at the proximal end of the device 76. The distal end of the obturator/cannula device 76 is equipped with a soft tip 80 that is for the obturating procedure. When this obturator/cannula device 76 is used it remains inserted in the patient's brain 16 for the entire procedure (whereas a separate obturator is removed after the pathway to the clot is established). Because the soft tip 80 of the obturator/cannula device 76 remains in-situ for the duration of the surgical procedure, this device 76 is equipped with a side exit port 82, the purpose of which will be described below. It is also preferred equipped with an ultrasound transmitter/receiver 84 and can also be equipped with a multi-sensor location (not shown) on the other side of the tip 80.

This combined obturator/cannula device 76 is preferably used with a specialized clot removal device 86 as seen in FIGS. 18a and 18b, which shows the specialized clot removal device 86 fully inserted and residing in the device 76 in FIG. 18a, and in a fully-inserted position in FIG. 18b, wherein the tip 80 is in an extended and articulated position. As shown in FIG. 19, this combined obturator/cannula device 76 and specialized clot removal device 86 can be inserted together through the alignment bushing 24, or the combined obturator/cannula device 76 can be inserted first through the alignment bushing 24, and then the specialized clot removal device 76 inserted once the device 76 has been partially or fully inserted into the patient's brain 16.

Once the two devices 76 and 86 have been inserted into the to-be-removed clot 18, the distal tip 88 of the device 86 is articulated by the surgeon to extend laterally outwardly from the exit port 82. The surgeon can then rotate the combined devices 76 and 86 to cause the extended tip 88 to rotate within the clot 18. As shown in FIG. 20a, the distal tip 88 is facing posteriorly in the clot 18, and in FIG. 20b, it is facing anteriorly within the clot. This laterally-extending and rotatable tip 88 allows for access to and viewing of the entire clot 18, and then more effective removal of the clot 18.

Another alternative preferred embodiment of a combined obturator/cannula device 90 is shown in FIGS. 21a and 21b in two perspective views. This device 90 eliminates the need for the peel-away sheath 52 and for a separate obturator 50. This device 90 is preferably equipped with transmitter/receiving port 92, and has a distal tip 94 that can be articulated such that once inserted into the clot 18, the tip 94 can be brought into a curved position by the surgeon. As this device 90 will also act as the obturator device, the tip 94 will preferably be of a soft material. FIGS. 22 and 23 show more detail as to the cannula portion of combined obturator/cannula device 90. As shown there, the distal tip 94 of the combined obturator/cannula device 90 will include the following elements: a visual scope 96 located that the very distal end of the tip 94; multiple suction/evacuation channels 98 for removal of clot 18 material; multiple irrigation channels 100 for delivering fluid into the clot site; and a functional channel 102 that can used for other devices as described above. FIGS. 24, 25 and 26 show this alternative preferred embodiment of a combined obturator/cannula device 90 positioned such that the distal tip 94 is located within the clot 18 upon insertion in the un-articulated state (FIG. 24), then with the distal tip 94 in an articulated state, facing posteriorly (FIG. 25), and then with the distal tip 94 in an articulated state, facing anteriorly (FIG. 25). By rotating the shaft of the device 90 (which can be rotated 360 degrees), the surgeon can view the entire clot.

There are numerous other additional elements that can added to the channels in the cannula. One such additional element is shown in FIG. 27, which is a high-speed, helical, auger type device 104 situated in a suction/evacuation channel 98. During the suction activity, this high-speed auger device 104 can be caused by the surgeon to rotate in so as to both aid in breaking up the clot material and forcing the blot material up and out of the suction/evacuation channel 98.

Details of the preferred integrated clot and tumor removal apparatuses, systems and methods have been detailed above. Other modifications, variations, and options are available and will be known to those of skill in the art. The scope and protection of the inventive concepts and embodiments shown, depicted and described herein are not limited to the embodiments, but are of the full scope and breadth of the claims. Nothing stated above is intended to be, or should be interpreted to be, a limitation that is incorporated by reference into the following claims.

What is claimed is:

1. An intracranial clot locating, evaluation and removal system comprising:
a) a control unit for controlling at least the functions of suction for removal of the clot and irrigation of the intracranial clot location;
b) an elongate obturator encased in a peel-away sheath having a desired interior and exterior diameter, such that after the obturator has been fully inserted into the proper intracranial position, said obturator can be removed, leaving said sheath in place creating a stable pathway to the intracranial clot location;
c) an elongate cannula sized and shaped to have an exterior diameter that mates with the interior diameter of said sheath such that said cannula can be inserted easily and snugly into said sheath to a position wherein the distal end of said cannula has access to said clot;
d) said cannula having multiple channels extending the length of said cannula, one or more of said channels being used for introduction of a clot removal device, for evacuation of the clot material, and for irrigation of the intracranial clot location;
e) a clot removal device operatively attached to said control unit, said clot removal device having body and a distal tip portion, said device being sized and shaped to be insertable through one of said channels within said cannula to a position wherein said distal tip portion of said cannula has access to said intracranial clot; and
f) said distal tip portion of said cannula being capable of articulation relative to said body such that said distal tip portion can be moved to a position that is at an angle relative to the longitudinal centerline of said body.

2. The system of claim 1, wherein said distal tip portion of said cannula includes a visual scope that produces a real time video image, and a video monitor that displays the video image relayed from said visual scope, thereby allowing the surgeon to obtain a 360-degree view of said intracranial clot and clot location before and after removal of said clot by rotation of said distal tip portion of said cannula.

3. The system of claim 1 in which said cannula or said clot removal device includes means for monitoring fluid pressure within said clot location.

4. The system of claim 1 in which said cannula or said clot removal device includes means for monitoring temperature within said clot location.

5. The system of claim 1 in which said cannula or said clot removal device includes means for monitoring oxygen levels within said clot location.

6. The system of claim 1 in which said cannula includes a channel for insertion of a device used to cauterize brain blood vessels during and after removal of said clot.

7. An elongate combination obturator and clot removal device, said device having a hollow elongate body with an atraumatic distal tip which functions as an obturator for gaining access to said intracranial clot; said elongate body have an exit port in close proximity to said distal tip; said hollow elongate body having a clot removal device with an articulating distal tip that can be caused by the surgeon to extend outwardly from said exit port.
